(19) United States
(12) Patent Application Publication
(10) Pub. No.: US 2023/0250426 A1
(43) Pub. Date: Aug. 10, 2023

(54) METHOD FOR TREATING AND PROGNOSING CANCER LIKE GLIOBLASTOMA

(71) Applicants: INSERM (INSTITUT NATIONAL DE LA SANTÉ ET DE LA RECHERCHE MÉDICALE), Paris (FR); ECOLE CENTRALE DE NANTES, Nantes (FR); INSTITUT DE CANCÉROLOGIE DE L'OUEST, Angers (FR); UNIVERSITÉ DE NANTES, Nantes (FR)

(72) Inventors: Pierre-François CARTRON, Nantes (FR); Gwenola BOUGRAS-CARTRON, Nantes (FR); Aurélien SERANDOUR, Nantes (FR); Joséphine BRIAND, Portland, OR (US)

(21) Appl. No.: 18/001,284

(22) PCT Filed: Jun. 9, 2021
(86) PCT No.: PCT/EP2021/065489

§ 371 Date: Dec. 9, 2022

(30) Foreign Application Priority Data
Jun. 10, 2020 (EP) 20305630.4

(51) Int. Cl. C12N 15/113; A61P 35/00; A61P 25/00; C12Q 1/6886

(52) U.S. Cl. CPC C12N 15/113; A61P 35/00; A61P 25/00; C12Q 1/6886; C12N 2310/141; C12N 2310/333; C12N 2310/3521; C12Q 2600/178; C12Q 2600/158; C12Q 2600/118; C12Q 2600/154

(57) ABSTRACT

The present invention relates the treatment and prognostic of cancer like glioblastoma. Here, the inventors focused their study on the impact of presence of N6-adenosine methylation in miRNA-200b-3p in samples of patients suffering from glioblastoma multiforme (GBM). Their study was particularly focused on the impact of miRNA-200b-3p and its adenosine methylation on the expression of XIAP. XIAP acts as an anti-apoptotic protein via the inhibition of caspase-3 and -7 activation and high XIAP expression is associated with a poor survival in several solid tumors. Thus, the miR-200b-3p-mediated repression of XIAP mRNA expression appears as a mechanism governing the caspase-3 and -7 activity and the apoptosis. In theory, in the presence of miR-200b-3p, XIAP mRNA expression is repressed and caspase-3 and -7 can be activated to promote apoptosis. Thus, the present invention relates to an in vitro method for determining the prognosis of the survival time of a patient suffering from a cancer comprising the steps consisting of i) determining the expression level of the miR-200b-3p and/or the N6-adenosine methylated miRNA-200b-3p (miR-200b-3p m6A) in a sample from said patient and to the N6-adenosine methylated miRNA-200b-3p (miR-200b-3p m6A) for use in the treatment of a cancer in a subject in need thereof.

### FIELD OF THE INVENTION

The present invention relates to an in vitro method for determining the prognosis of the survival time of a patient suffering from a cancer comprising the steps consisting of i) determining the expression level of the miR-200b-3p and/or the N6-adenosine methylated miRNA-200b-3p (miR-200b-3p m6A) in a sample from said patient and to the N6-adenosine methylated miRNA-200b-3p (miR-200b-3p m6A) for use in the treatment of a cancer in a subject in need thereof.

### BACKGROUND OF THE INVENTION

MicroRNA (miRNA) are short non-coding RNAs that regulate protein expression towards their function of translational repressor. Thus, miRNA are crucial regulators of many cellular processes including proliferation, apoptosis, immunogenicity, development and differentiation. miRNA biogenesis can be epigenetically regulated in both physiological and pathological conditions toward the DNA methylation of miRNA genes. Wang et al. report that the expression of approximately 50% of miRNA genes is putatively regulated by DNA methylation since they are associated with CpG islands [1]. A variety of DNA methylation-specific methyl-CpG-binding domain proteins (MBD) were also found to transcriptionally regulate miRNA genes [2]. Finally, Malumbres et al. also report that the miRNA genes expression is also regulated through histone modifications, such as lysine methylation and acetylation [3].

Several publications report that chemical modifications can occur in miRNA and that these modifications regulate the miRNA processing or functionality. Among these modifications, some can affect the phosphate at the 5′-end of miRNA. Thus, Xhemalce et al. (2012) report that the BCDIN3D-mediated phospho-dimethylation of miRNAs (such as pre-miR-145) negatively regulates the miRNA maturation and impacts on the tumorigenic phenotype [4]. Other chemical modifications of miRNA affect the internal bases of miRNA. Alarcón et al. (2015) and Berulava et al. (2015) report that miRNA can be adenosine methylated and that the presence of this methylation promotes the initiation of miRNA biogenesis and increases the stability of adenosine methylated miRNAs, respectively [5] [6]. Konno et al. (2019) also report that miRNA can be adenosine methylated [7]. Besides, in this report, authors introduced the idea that the adenosine methylation of miRNA can be used as biomarker for the diagnosis of early-stage cancer. Pandolfini et al. (2019) report that miRNA can be guanosine methylated and that this methylation inhibits the miRNA maturation [8]. Recently, our laboratory published that miRNAs can be cytosine methylated and that the presence of this methylation represses the miRNA function [9].

Several enzymes catalyze these base modifications: METTL1 (Methyltransferase-like protein 1, Uniprot Q9UBP6) and DNMT3A (DNA (cytosine-5)-methyltransferase 3A, Uniprot Q9Y6K1) are defined to promote the guanosine and cytosine methylation of miRNAs, respectively [8] [9]. The complex METTL3-WTAP-METTL14 is described as a miRNA adenosine methylase or writer, while FTO (Fat mass and obesity-associated protein, Uniprot Q9C0B1) and ALKBH5 (Alkylated DNA repair protein alkB homolog 5, Uniprot Q6P6C2) are described as miRNA adenosine demethylases or erasers, [6] [10] [11] [12] [5] [13].

Interestingly, these two enzymes are alpha-KetoGlutarate-dependent (aKG) suggesting that the adenosine methylation of miRNA can be regulated by the intracellular level of αKG. αKG is a Krebs cycle metabolite. It is formed from isocitrate by oxidative decarboxylation catalyzed by IDH proteins and plays a key role in multiple metabolic and cellular pathways via its co-substrate role of several enzymes such as FTO and ALKBH5 [14]. Thus, in theory, high level of αKG should increase the FTO activity and should promote a decrease of adenosine methylation of miRNA.

Despite these undeniable advances, further studies of the molecular mechanisms governing the chemical modifications of miRNA in a tumor context is required in order to increase the understanding of the role played by these modifications in tumors.

### SUMMARY OF THE INVENTION

Here, the inventors focused their study on the impact of presence of N6-adenosine methylation in miRNA-200b-3p in samples of patients suffering from glioblastoma multiforme (GBM). Their study was particularly focused on the impact of miRNA-200b-3p and its adenosine methylation on the expression of XIAP (X-linked inhibitor of apoptosis protein, Uniprot P98170). XIAP acts as an anti-apoptotic protein via the inhibition of caspase-3 and -7 activation and high XIAP expression is associated with a poor survival in several solid tumors [15] [16]. Thus, the miR-200b-3p-mediated repression of XIAP mRNA expression appears as a mechanism governing the caspase-3 and -7 activity and the apoptosis. In theory, in the presence of miR-200b-3p, XIAP mRNA expression is repressed and caspase-3 and -7 can be activated to promote apoptosis. However, in absence or inactivation of the miR-200b-3p, XIAP is expressed, blocks caspase-3 and -7 activation and therefore inhibits apoptosis. This is why it is important to combine miR-200b-3p expression study to its action capacity.

Thus, the present invention relates in vitro method for determining the prognosis of the survival time of a patient suffering from a cancer comprising the steps consisting of i) determining the expression level of the miR-200b-3p and/or the N6-adenosine methylated miRNA-200b-3p (miR-200b-3p m6A) in a sample from said patient, ii) comparing said expression level with a predetermined reference value and iii) providing a good prognosis when the expression level of miR-200b-3p m6A is inferior to 10% and the expression level of the miR-200b-3 is higher than the predetermined reference value and a poor prognosis when the expression level of miR-200b-3p m6A is inferior to 10% and the expression level of the miR-200b-3p is lower than the predetermined reference value or when the expression level of miR-200b-3p m6A is superiors to 10%. The invention also relates to the N6-adenosine methylated miRNA-200b-3p (miR-200b-3p m6A) for use in the treatment of a cancer in a subject in need thereof. Particularly, the invention is defined by its claims.

### DETAILED DESCRIPTION OF THE INVENTION

### Prognostic Method

The first aspect of the invention relates to an in vitro method for determining the prognosis of the survival time of a patient suffering from a cancer comprising the steps consisting of i) determining the expression level of the miR-200b-3p and/or the N6-adenosine methylated miRNA-200b-3p (miR-200b-3p m6A) in a sample from said patient, ii) comparing said expression level with a predetermined reference value and iii) providing a good prognosis when the expression level of miR-200b-3p m6A is inferior to 10% and the expression level of the miR-200b-3 is higher than the predetermined reference value and a poor prognosis when the expression level of miR-200b-3p m6A is inferior to 10% and the expression level of the miR-200b-3p is lower than the predetermined reference value or when the expression level of miR-200b-3p m6A is superiors to 10%.

Particularly, the invention relates to an in vitro method for determining the prognosis of the survival time of a patient suffering from a cancer comprising the steps consisting of i) determining the expression level of the miR-200b-3p and the N6-adenosine methylated miRNA-200b-3p (miR-200b-3p m6A) in a sample from said patient, ii) comparing said expression level with a predetermined reference value and iii) providing a good prognosis when the expression level of miR-200b-3p m6A is inferior to 10% and the expression level of the miR-200b-3 is higher than the predetermined reference value and a poor prognosis when the expression level of miR-200b-3p m6A is inferior to 10% and the expression level of the miR-200b-3p is lower than the predetermined reference value.

In one embodiment, the cancer may be any solid or liquid cancer. Typically, the cancer may be selected from the group consisting of bile duct cancer (e.g. periphilar cancer, distal bile duct cancer, intrahepatic bile duct cancer), bladder cancer, bone cancer (e.g. osteoblastoma, osteochrondroma, hemangioma, chondromyxoid fibroma, osteosarcoma, chondrosarcoma, fibrosarcoma, malignant fibrous histiocytoma, giant cell tumor of the bone, chordoma, lymphoma, multiple myeloma), brain and central nervous system cancer (e.g. meningioma, astocytoma, oligodendrogliomas, glioblastoma, ependymoma, gliomas, medulloblastoma, ganglioglioma, Schwannoma, germinoma, craniopharyngioma), breast cancer (e.g. ductal carcinoma in situ, infiltrating ductal carcinoma, infiltrating, lobular carcinoma, lobular carcinoma in, situ, gynecomastia), Castleman disease (e.g. giant lymph node hyperplasia, angiofollicular lymph node hyperplasia), cervical cancer, colorectal cancer, endometrial cancer (e.g. endometrial adenocarcinoma, adenocanthoma, papillary serous adnocarcinroma, clear cell), esophagus cancer, gallbladder cancer (mucinous adenocarcinoma, small cell carcinoma), gastrointestinal carcinoid tumors (e.g. choriocarcinoma, chorioadenoma destruens), Hodgkin's disease, non-Hodgkin's lymphoma, Kaposi's sarcoma, kidney cancer (e.g. renal cell cancer), laryngeal and hypopharyngeal cancer, liver cancer (e.g. hemangioma, hepatic adenoma, focal nodular hyperplasia, hepatocellular carcinoma), lung cancer (e.g. small cell lung cancer, non-small cell lung cancer), mesothelioma, plasmacytoma, nasal cavity and paranasal sinus cancer (e.g. esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma (e.g. embryonal rhabdomyosarcoma, alveolar rhabdomyosarcoma, pleomorphic rhabdomyosarcoma), salivary gland cancer, skin cancer (e.g. melanoma, nonmelanoma skin cancer), stomach cancer, testicular cancer (e.g. seminoma, nonseminoma germ cell cancer), thymus cancer, thyroid cancer (e.g. follicular carcinoma, anaplastic carcinoma, poorly differentiated carcinoma, medullary thyroid carcinoma, thyroid lymphoma), vaginal cancer, vulvar cancer, and uterine cancer (e.g. uterine leiomyosarcoma).

In a particular embodiment, the glioblastoma is a glioblastoma multiforme (GBM).

Typically, the sample according to the invention may be a blood, plasma, serum sample or a cancer biopsy. In a particular embodiment, said sample is a glioblastoma biopsy.

According to the invention, the term “patient” or “patient” denotes a human with a cancer and particularly a GBM.

As used herein, the term “miR-200b-3p” (miRBase database ID number: MIMAT0000318) denotes a member of the tumor suppressive miRNA family, miR-200.

As used herein the term “N6-adenosine methylated miRNA-200b-3p” or “miR-200b-3p m6A” denotes the presence of a methylation on the second last adenosine in 3′ in the miRNA-200b-3p (in bold and underline below).

Acid nucleic sequence of miR-200b-3p (SEQ ID NO: 1) is: UAAUACUGCCUGGUAAUGAUGA

As used herein, the term “level of miR-200b-3p m6A inferior to 10%” or “level of miR-200b-3p m6A superior to 10%” denotes the percentage of miR-200b-3p m6A compared to the total of miR-200b-3p. Thus, for example, a level of miR-200b-3p m6A superior to 10% denotes that more than 10% of the miR-200b-3p is methylated.

In another embodiment, the invention relates to an in vitro method for determining the prognosis of the overall survival (OS) of a patient suffering from a cancer comprising the steps consisting of i) determining the expression level of the miR-200b-3p and/or the N6-adenosine methylated miRNA-200b-3p (miR-200b-3p m6A) in a sample from said patient, ii) comparing said expression level with a predetermined reference value and iii) providing a good prognosis when the expression level of miR-200b-3p m6A is inferior to 10% and the expression level of the miR-200b-3 is higher than the predetermined reference value and a poor prognosis when the expression level of miR-200b-3p m6A is inferior to 10% and the expression level of the miR-200b-3p is lower than the predetermined reference value or when the expression level of miR-200b-3p m6A is superiors to 10%.

As used herein, the term “Overall survival (OS)” denotes the percentage of people in a study or treatment group who are still alive for a certain period of time after they were diagnosed with or started treatment for a disease, such as a cancer (according to the invention).

As used herein, the term “Good Prognosis” denotes a patient with more than 50% chance of survival for the next 3 years after the treatment.

In one embodiment and according to the methods of the invention, the determination of the expression level of the miR of the invention may be determined before or after the beginning of a treatment of the patient.

In another embodiment, the patient affected with a cancer and particularly a glioblastoma is mainly treated with a standard treatment consisting of maximal surgical resection, radiotherapy, and concomitant adjuvant chemotherapy with temozolomide.

The term “determining the expression level of” as used above includes qualitative and/or quantitative detection (measuring levels) with or without reference to a control. Typically expression level of the miR of the invention may be measured for example by RNA-immunoprecipitation, Cross-linking immunoprecipitation, qRT-PCR performed and all RNA sequencing methods on the sample.

The “reference value” may be a healthy subject, i.e. a subject who does not suffer from any cancer and particularly glioblastoma. Particularly, said control is a not a healthy subject.

In the case of detection of the miR of the invention, the term “expression level of miR-200b-3p and/or the N6-adenosine methylated miRNA-200b-3p (miR-200b-3p m6A)” refers to an amount or a concentration of the miR methylated or not. Typically, a level of miR expression can be expressed in units such as transcripts per cell or nanograms per microgram of tissue. Alternatively, relative units can be employed to describe an expression level.

Measuring the expression level of a miR can be performed by a variety of techniques well known in the art.

Methods for determining the quantity of miR are well known in the art. For example the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted miR is then detected by hybridization (e. g., Northern blot analysis, in situ hybridization) and/or amplification (e.g., RT-PCR).

Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the miR of interest herein find utility as hybridization probes or amplification primers. It is understood that such nucleic acids need not be identical, but are typically at least about 80% identical to the homologous region of comparable size, more particularly 85% identical and even more particularly 90-95% identical. In certain embodiments, it will be advantageous to use nucleic acids in combination with appropriate means, such as a detectable label, for detecting hybridization.

Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook-A Guide to Fluorescent Probes and Labeling Technologies). Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl) aminonaphthalene-1-sulfonic acid (EDANS), 4-amino-N-[3 vinylsulfonyl)phenyl]naphthalimide-3,5 disulfonate (Lucifer Yellow VS), N-(4-anilino-1-naphthyl)maleimide, antllranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diarninidino-2-phenylindole (DAPI); 5′,5″dibromopyrogallol-sulfonephthalein (Bromopyrogallol Red); 7-diethylamino-3 (4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulforlic acid; 5-[dimethylamino] naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6diclllorotriazin-2-yDarninofluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 mn (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6, 130, 101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT™ (obtained, for example, from Life Technologies (QuantumDot Corp, Invitrogen Nanocrystal Technologies, Eugene, Oreg.); see also, U.S. Pat. Nos. 6,815,064; 6,682,596; and 6,649, 138). Semiconductor nanocrystals are microscopic particles having size-dependent optical and/or electrical properties. When semiconductor nanocrystals are illuminated with a primary energy source, a secondary emission of energy occurs of a frequency that corresponds to the handgap of the semiconductor material used in the semiconductor nanocrystal. This emission can be detected as colored light of a specific wavelength or fluorescence. Semiconductor nanocrystals with different spectral characteristics are described in e.g., U.S. Pat. No. 6,602,671. Semiconductor nanocrystals that can be coupled to a variety of biological molecules (including dNTPs and/or nucleic acids) or substrates by techniques described in, for example, Bruchez et al., Science 281:20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Publication No. 99/26299 (published May 27, 1999). Separate populations of semiconductor nanocrystals can be produced that are identifiable based on their different spectral characteristics. For example, semiconductor nanocrystals can be produced that emit light of different colors hazed on their composition, size or size and composition. For example, quantum dots that emit light at different wavelengths based on size (565 mn, 655 mn, 705 mn, or 800 mn emission wavelengths), which are suitable as fluorescent labels in the probes disclosed herein are available from Life Technologies (Carlshad, Calif.).

Additional labels include, for example, radioisotopes (such as 3H), metal chelates such as DOTA and DPTA chelates of radioactive or paramagnetic metal ions like Gd3+, and liposomes.

Detectable labels that can be used with nucleic acid molecules also include enzymes, for example horseradish peroxidase, alkaline phosphatase, acid phosphatase, glucose oxidase, beta-galactosidase, beta-glucuronidase, or beta-lactamase.

Alternatively, an enzyme can be used in a metallographic detection scheme. For example, silver in situ hyhridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redoxactive agent reduces the metal ion, causing it to form a detectable precipitate. (See, for example, U.S. Patent Application Publication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water soluble metal ion, an oxidizing agent and a reducing agent, again to form a detectable precipitate. (See, for example, U.S. Pat. No. 6,670,113).

Probes made using the disclosed methods can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat antiavidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pirlkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am.1. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/01 17153.

It will be appreciated by those of skill in the art that by appropriately selecting labelled probe-specific binding agent pairs, multiplex detection schemes can be produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can be labelled with a first hapten, such as biotin, while a second probe that corresponds to a second target sequence can be labelled with a second hapten, such as DNP. Following exposure of the sample to the probes, the bound probes can be detected by contacting the sample with a first specific binding agent (in this case avidin labelled with a first fluorophore, for example, a first spectrally distinct QUANTUM DOT®, e.g., that emits at 585 mn) and a second specific binding agent (in this case an anti-DNP antibody, or antibody fragment, labelled with a second fluorophore (for example, a second spectrally distinct QUANTUM DOT®, e.g., that emits at 705 mn). Additional probes/binding agent pairs can be added to the multiplex detection scheme using other spectrally distinct fluorophores. Numerous variations of direct, and indirect (one step, two step or more) can be envisioned, all of which are suitable in the context of the disclosed probes and assays.

Probes typically comprise single-stranded nucleic acids of between 10 to 1000 nucleotides in length, for instance of between 10 and 800, more particularly of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they particularly hybridize under high stringency hybridization conditions (corresponding to the highest melting temperature Tm, e.g., 50% formamide, 5x or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A particular kit also includes the components necessary to determine if amplification has occurred. The kit may also include, for example, PCR buffers and enzymes; positive control sequences, reaction control primers; and instructions for amplifying and detecting the specific sequences.

In a particular embodiment, the methods of the invention comprise the steps of providing total miR extracted from cumulus cells and subjecting the miR to amplification and hybridization to specific probes, more particularly by means of a quantitative or semi-quantitative RT-PCR.

In another particular embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

Expression level of a gene may be expressed as absolute expression level or normalized expression level. Typically, expression levels are normalized by correcting the absolute expression level of a gene by comparing its expression to the expression of a gene that is not a relevant for determining the cancer stage of the patient, e.g., a housekeeping gene that is constitutively expressed. Suitable genes for normalization include housekeeping genes such as the actin gene ACTB, ribosomal 18S gene, GUSB, PGK1 and TFRC. According to the invention the housekeeping genes used were GAPDH, GUSB, TBP and ABL1. This normalization allows the comparison of the expression level in one sample, e.g., a patient sample, to another sample, or between samples from different sources.

Typically, a “threshold value”, “threshold level”, “reference value” or “cut-off value” can be determined experimentally, empirically, or theoretically. A threshold value can also be arbitrarily selected based upon the existing experimental and/or clinical conditions, as would be recognized by a person of ordinary skilled in the art. Particularly, the person skilled in the art may compare the expression levels of the miR of the invention obtained according to the method of the invention with a defined threshold value.

Particularly, said threshold value is the mean expression level of the miR of the invention of a population of healthy individuals. As used herein, the term “healthy individual” denotes a human which is known to be healthy, i.e. which does not suffer from a cancer and in particular from a glioblastoma and does not need any medical care.

Typically, the skilled person in the art may determine the expression level of the miR of the invention in a biological sample, particularly a biopsy of a glioblastoma cancer for example, of 100 individuals known to be healthy or not. The mean value of the obtained expression levels is then determined, according to well-known statistical analysis, so as to obtain the mean expression level of the miR of the invention. Said value is then considered as being normal and thus constitutes a threshold value. By comparing the expression levels of the miR of the invention to this threshold value, the physician is then able to classify and prognostic the cancer.

Accordingly, the physician would be able to adapt and optimize appropriate medical care of a patient in a critical and life-threatening condition suffering from cancer. The determination of said prognosis is highly appropriate for follow-up care and clinical decision making.

The present invention also relates to kits useful for the methods of the invention, comprising means for detecting the miR of the invention.

According to the invention, the kits of the invention may comprise an anti-DNMT3A protein antibody and an anti-ISGF3γ; and another molecule coupled with a signalling system which binds to said DNMT3A/ISGF3γ antibodies or any molecule which bind to the mRNA of DNMT3A ISGF3γ genes like a probe.

Typically, the antibodies or combination of antibodies are in the form of solutions ready for use. In one embodiment, the kit comprises containers with the solutions ready for use. Any other forms are encompassed by the present invention and the man skilled in the art can routinely adapt the form to the use in immunohistochemistry.

### Therapeutic Method

A second aspect of the invention relates to the N6-adenosine methylated miRNA-200b-3p (miR-200b-3p m6A) for use in the treatment of a cancer in a subject in need thereof.

According to the invention, the miR-200b-3p m6A has the nucleic acid sequence SEQ ID NO:1 with a methylation on the second last nucleic acid on 3′.

In one embodiment, the invention relates to the miR-200b-3p m6A as a prodrug for use in the treatment of a cancer in a subject in need thereof.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

The invention also relates to i) the miR-200b-3p m6A according to the invention and ii) a conventional treatment used to treat cancer, as a combined preparation for simultaneous, separate or sequential for use in the treatment of a cancer in a subject in need thereof.

As used herein, the terms “conventional treatment used to treat cancer” denote any compounds, combination of compounds, combination of chemotherapeutic treatment and radiotherapeutic agent and combination of chemotherapeutic treatment and radiation which may be used for the treatment of cancer. For example, in the case of the treatment of glioblastoma, the conventional treatment may the use of a combination of the temozolomide and radiation.

Thus, the invention also relates to i) the miR-200b-3p m6A according to the invention and ii) a chemotherapeutic agent and iii) a radiotherapy or a radiotherapeutic agent, as a combined preparation for simultaneous, separate or sequential for use in the treatment of a cancer in a subject in need thereof.

As used herein, “radiotherapy” may consist of gamma-radiation, X-ray radiation, electrons or photons, external radiotherapy or curitherapy.

As used herein, the term “radiotherapeutic agent”, is intended to refer to any radiotherapeutic agent known to one of skill in the art to be effective to treat or ameliorate cancer, without limitation. For instance, the radiotherapeutic agent can be an agent such as those administered in brachytherapy or radionuclide therapy. Such methods can optionally further comprise the administration of one or more additional cancer therapies, such as, but not limited to, chemotherapies, and/or another radiotherapy.

According to the invention, the chemotherapeutic agent may be the temozolomide, 5-aza-2′-deoxycytidine, Theaflavin 3, 3′-digallate, zebularine, decitabine, 4-amino-N-(4-aminophenyl), benzamide analogues of quinoline-based SGI-1027 (PMID: 24678024 or 23294304.

In one embodiment, the cancer according to the invention is a glioblastoma.

In one embodiment, the invention relates to i) the miR-200b-3p m6A according to the invention and ii) a chemotherapeutic agent and iii) a radiotherapy, as a combined preparation for simultaneous, separate or sequential for use in the treatment of a glioblastoma in a subject in need thereof.

In a particular embodiment, the invention relates to i) the miR-200b-3p m6A according to the invention and ii) the temozolomide and iii) a radiotherapy, as a combined preparation for simultaneous, separate or sequential for use in the treatment of a glioblastoma in a subject in need thereof.

According to the invention, the term “subject” denotes a mammal, such as a rodent, a feline, a canine, and a primate. In some embodiments, the subject is a human. In some embodiments, the subject is a human infant. Particularly, the subject denotes an human with a cancer and particularly a GBM.

In one embodiment and according to the method of treatment, the cancer may be any solid or liquid cancer. Typically, the cancer may be selected from the group consisting of bile duct cancer (e.g. periphilar cancer, distal bile duct cancer, intrahepatic bile duct cancer), bladder cancer, bone cancer (e.g. osteoblastoma, osteochrondroma, hemangioma, chondromyxoid fibroma, osteosarcoma, chondrosarcoma, fibrosarcoma, malignant fibrous histiocytoma, giant cell tumor of the bone, chordoma, lymphoma, multiple myeloma), brain and central nervous system cancer (e.g. meningioma, astocytoma, oligodendrogliomas, glioblastoma, ependymoma, gliomas, medulloblastoma, ganglioglioma, Schwannoma, germinoma, craniopharyngioma), breast cancer (e.g. ductal carcinoma in situ, infiltrating ductal carcinoma, infiltrating, lobular carcinoma, lobular carcinoma in, situ, gynecomastia), Castleman disease (e.g. giant lymph node hyperplasia, angiofollicular lymph node hyperplasia), cervical cancer, colorectal cancer, endometrial cancer (e.g. endometrial adenocarcinoma, adenocanthoma, papillary serous adnocarcinroma, clear cell), esophagus cancer, gallbladder cancer (mucinous adenocarcinoma, small cell carcinoma), gastrointestinal carcinoid tumors (e.g. choriocarcinoma, chorioadenoma destruens), Hodgkin's disease, non-Hodgkin's lymphoma, Kaposi's sarcoma, kidney cancer (e.g. renal cell cancer), laryngeal and hypopharyngeal cancer, liver cancer (e.g. hemangioma, hepatic adenoma, focal nodular hyperplasia, hepatocellular carcinoma), lung cancer (e.g. small cell lung cancer, non-small cell lung cancer), mesothelioma, plasmacytoma, nasal cavity and paranasal sinus cancer (e.g. esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma (e.g. embryonal rhabdomyosarcoma, alveolar rhabdomyosarcoma, pleomorphic rhabdomyosarcoma), salivary gland cancer, skin cancer (e.g. melanoma, nonmelanoma skin cancer), stomach cancer, testicular cancer (e.g. seminoma, nonseminoma germ cell cancer), thymus cancer, thyroid cancer (e.g. follicular carcinoma, anaplastic carcinoma, poorly differentiated carcinoma, medullary thyroid carcinoma, thyroid lymphoma), vaginal cancer, vulvar cancer, and uterine cancer (e.g. uterine leiomyosarcoma).

In a particular embodiment, the glioblastoma is a glioblastoma multiforme (GBM).

Particularly, the cancer is a cancer which express the enzymes FTO (Fat mass and obesity-associated protein, Uniprot Q9C0B1) and αKG (Alkylated DNA repair protein alkB homolog 5, Uniprot Q6P6C2) like the GBM.

Particularly, the cancer is a cancer with no mutations in IDH1 (Isocitrate dehydrogenase 1).

Another object of the invention relates to a method for treating cancer comprising administrating to a subject in need thereof a therapeutically effective amount of the N6-adenosine methylated miRNA-200b-3p (miR-200b-3p m6A).

### Therapeutic Composition

Another object of the invention relates to a therapeutic composition comprising the N6-adenosine methylated miRNA-200b-3p (miR-200b-3p m6A) according to the invention for use in the treatment of cancer in a subject in need thereof.

In one embodiment, the invention relates to a therapeutic composition comprising the N6-adenosine methylated miRNA-200b-3p (miR-200b-3p m6A) according to the invention for use in the treatment of glioblastoma in a subject in need thereof.

Any therapeutic agent of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, parenteral, intraocular, intravenous, intramuscular or subcutaneous administration and the like.

Particularly, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

In addition, other pharmaceutically acceptable forms include, e.g. tablets or other solids for oral administration; time release capsules; and any other form currently can be used.

Pharmaceutical compositions of the present invention may comprise a further therapeutic active agent. The present invention also relates to a kit comprising a compound according to the invention and a further therapeutic active agent.

In one embodiment said therapeutic active agent may be an anti-cancer agent.

Anti-cancer agents may be Melphalan, Vincristine (Oncovin), Cyclophosphamide (Cytoxan), Etoposide (VP-16), Doxorubicin (Adriamycin), Liposomal doxorubicin (Doxil) and Bendamustine (Treanda).

Others anti-cancer agents may be for example cytarabine, anthracyclines, fludarabine, gemcitabine, capecitabine, methotrexate, taxol, taxotere, mercaptopurine, thioguanine, hydroxyurea, cyclophosphamide, ifosfamide, nitrosoureas, platinum complexes such as cisplatin, carboplatin and oxaliplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epimbicm, 5-fluorouracil, taxanes such as docetaxel and paclitaxel, leucovorin, levamisole, irinotecan, estramustine, etoposide, nitrogen mustards, BCNU, nitrosoureas such as carmustme and lomustine, vinca alkaloids such as vinblastine, vincristine and vinorelbine, imatimb mesylate, hexamethyhnelamine, topotecan, kinase inhibitors, phosphatase inhibitors, ATPase inhibitors, tyrphostins, protease inhibitors, inhibitors herbimycm A, genistein, erbstatin, and lavendustin A. In one embodiment, additional anticancer agents may be selected from, but are not limited to, one or a combination of the following class of agents: alkylating agents, plant alkaloids, DNA topoisomerase inhibitors, anti-folates, pyrimidine analogs, purine analogs, DNA antimetabolites, taxanes, podophyllotoxin, hormonal therapies, retinoids, photosensitizers or photodynamic therapies, angiogenesis inhibitors, antimitotic agents, isoprenylation inhibitors, cell cycle inhibitors, actinomycins, bleomycins, MDR inhibitors and Ca2+ ATPase inhibitors.

Additional anti-cancer agents may be selected from, but are not limited to, cytokines, chemokines, growth factors, growth inhibitory factors, hormones, soluble receptors, decoy receptors, monoclonal or polyclonal antibodies, mono-specific, bi-specific or multi-specific antibodies, monobodies, polybodies.

Additional anti-cancer agent may be selected from, but are not limited to, growth or hematopoietic factors such as erythropoietin and thrombopoietin, and growth factor mimetics thereof.

In the present methods for treating cancer the further therapeutic active agent can be an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoemanolamine, alizapride, azasetron, benzquinamide, bietanautine, bromopride, buclizine, clebopride, cyclizine, dunenhydrinate, diphenidol, dolasetron, meclizme, methallatal, metopimazine, nabilone, oxypemdyl, pipamazine, scopolamine, sulpiride, tetrahydrocannabinols, thiefhylperazine, thioproperazine and tropisetron. In a preferred embodiment, the antiemetic agent is granisetron or ondansetron.

In another embodiment, the further therapeutic active agent can be an hematopoietic colony stimulating factor. Suitable hematopoietic colony stimulating factors include, but are not limited to, filgrastim, sargramostim, molgramostim and epoietin alpha.

In still another embodiment, the other therapeutic active agent can be an opioid or non-opioid analgesic agent. Suitable opioid analgesic agents include, but are not limited to, morphine, heroin, hydromorphone, hydrocodone, oxymorphone, oxycodone, metopon, apomorphine, nomioiphine, etoipbine, buprenorphine, mepeddine, lopermide, anileddine, ethoheptazine, piminidine, betaprodine, diphenoxylate, fentanil, sufentanil, alfentanil, remifentanil, levorphanol, dextromethorphan, phenazodne, pemazocine, cyclazocine, methadone, isomethadone and propoxyphene. Suitable non-opioid analgesic agents include, but are not limited to, aspirin, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

In yet another embodiment, the further therapeutic active agent can be an anxiolytic agent. Suitable anxiolytic agents include, but are not limited to, buspirone, and benzodiazepines such as diazepam, lorazepam, oxazapam, chlorazepate, clonazepam, chlordiazepoxide and alprazolam.

In yet another embodiment, the further therapeutic active agent can be a checkpoint blockade cancer immunotherapy agent.

Typically, the checkpoint blockade cancer immunotherapy agent is an agent which blocks an immunosuppressive receptor expressed by activated T lymphocytes, such as cytotoxic T lymphocyte-associated protein 4 (CTLA4) and programmed cell death 1 (PDCD1, best known as PD-1), or by NK cells, like various members of the killer cell immunoglobulin-like receptor (KIR) family, or an agent which blocks the principal ligands of these receptors, such as PD-1 ligand CD274 (best known as PD-L1 or B7-H1).

Typically, the checkpoint blockade cancer immunotherapy agent is an antibody.

In some embodiments, the checkpoint blockade cancer immunotherapy agent is an antibody selected from the group consisting of anti-CTLA4 antibodies, anti-PD1 antibodies, anti-PDL1 antibodies, anti-PDL2 antibodies, anti-TIM-3 antibodies, anti-LAG3 antibodies, anti-IDO1 antibodies, anti-TIGIT antibodies, anti-B7H3 antibodies, anti-B7H4 antibodies, anti-BTLA antibodies, and anti-B7H6 antibodies.

The invention will be further illustrated by the following figures and examples. However, these examples and figures should not be interpreted in any way as limiting the scope of the present invention.

### FIGURES

FIG. 1: The N6-adenosine methylation of miR-200b-3p limits its translational repressor function toward anti-apoptotic players and confers poor prognosis in GBM patients.

A. Samples were stratified according to the miR-200b-3pexp and miR-200b-3p % m6A parameters in order to distinguish the 3 indicated groups. Each box represents a sample/patient. For each group, the average of XIAP expression was analyzed with Human XIAP ELISA Kit (Abcam, France) was calculated and represented on the graph.

B. Kaplan-Meier representation of survival curves for GBM patients those tumors are characterized by a miR-200b-3pm6A>10% or a miRNA-200b-3pexp-low and by a miR-200b-3pm6A<10% and a miRNA-200b-3pexp-high.

FIG. 2: The N6-adenosine methylation of miR-200b-3p selectively induces apoptosis in cancer cells and has an anti-tumor growth effect.

A. miR-200b-3p promotes cell death by itself in cancerous and non-cancerous cells (excepted neuron RN33b), while miR-200b-3b induced apoptosis by itself in U87 cells, only. The LDH-Cytotoxicity Assay Kit (Abcam, France) is used to estimate the cell death 24h after the m6A-miR-200b-3b incubation.

B. Impact of the adenosine-methylated form of miR-200b-3p on the tumor growth in mice model.

FIG. 3: miR-200b-3p could also be used as a therapeutic tool in other cancer types.

In cell lines transfected with m6A-miR-200b-3p, cell death is induced in several cancer cell line types, when cell lines are able to demethylate this miR.

### Example

Material & Methods

miRNA Extraction

miRNA extractions were performed using the NucleoSpin® miRNA kit (Macherey Nagel, France) according to the manufacturer's instructions.

miRNA and siRNA Transfection

Briefly, 6×105 cells were seeded in each well of 4-well plates. Transfection was performed using HiPerFect Transfection Reagents (Qiagen, France) and 10 ng miR (Qiagen, France) or 10 nm of Silencer® siRNA (Thermo Fisher, France), according to the manufacturer's recommendations. For siRNA controls, transfection control (HiPerfect Transfection Reagent only) and a negative control (Silencer® Negative control #1 siRNA) had been used. For miR controls, transfection control (HiPerfect Transfection Reagent only) and an oligo (miScript Inhibitor Negative Control; Qiagen, France) had been used.

Acellular METTL3 Methylation Assay

METTL3-including complexes were immunoprecipitated from cellular lysate obtained after sonication and the use of CHAPS buffer (40 mM HEPES, pH 7.4, 120 mM NaCl, 1% CHAPS, 1 mM EDTA, supplemented with protease and phosphatase inhibitors). Immunoprecipitations were performed using Catch and Release v2.0 Reversible Immunoprecipitation System (Merck, France) and anti-METTL3 (Abcam, France). IgG (Abcam, France) was used as control. Elutions from IP were performed using the non-denaturing Elution Buffer according to the manufacturer's instructions. Then 30 μL of elution were used in METTL3 enzymatic assay. METTL3 enzymatic assay was conducted in reaction buffer (20 mM Tris pH 7.5, 1 mM DTT, 0.01% Triton X-100, 40U/100 ml buffer RNaseOUT). The reaction mixture contained unmethylated mimic miR-200b-3p with biotin tag and SAM. Enzymatic assay reactions were incubated overnight at room temperature on shaker. After streptavidin isolation, the presence of N6-adenosine methylation was determined by dot blot. Dots were then incubated with anti-m6A and anti-adenosine (as loading control) antibodies overnight. For signal detection secondary HRP antibodies were used and signal was detected on ChemiDoc MP (Bio-Rad, France).

RNA-Immunoprecipitation for miRNA

For immunoprecipitation of RNA, two rounds using 5 μg of anti-m6A antibody (Abcam, France) and 5 μg of small RNA were performed. The reaction was carried out using Dynabeads Protein G Immunoprecipitation kit with some modifications (ThermoFisher Scientific, France) such as described by Berulava et al. (2015) [6]. As a control, immunoprecipitation was 15 performed using IgG (Abcam, France) instead of anti-m6A antibody. miRs obtained from m6A immunoprecipitation were reverse transcribed using miRScript II RT kit (Qiagen, France) and analyzed using the miScript miRNA PCR Array Human Cancer Pathway kit (Qiagen, France) according to the manufacturers' instructions. Fold enrichment was next calculated using Ct value obtained from RT-qPCR performed with input miR, IP-IgG and IP-m6A and the 2-ΔΔCt formula.

Cross-Linking Immunoprecipitation (CLIP)

CLIP were performed using RiboCluster Profiler RIP-Assay (CliniScience, France) from 10 millions per sample of UV crosslinked cells (150 mJ/cm2 of UVA (365 nm) according to the manufacturer's instructions. IP were performed in presence of 15 g of anti-GW182 (#RN033P, CliniScience, France) and anti-TNRC6B (#9913, Merck-Millipore, France) for overnight at 4° C.

Quantitative PCR of miRNA

For miRNA expression analysis and detection from product of RIP performed with anti-m6A antibody, RNA was reverse transcribed using miRScript II RT kit and analyzed by qPCR with the miScript SYBR Green PCR Kit using the specific hsa-miR miScript Primer Assays (Qiagen, France) according to the manufacturers' instructions.

ELISA

Proteins extracts were obtained by using RIPA Lysis and Extraction Buffer (Thermo Scientific, France) in accordance with the manufacturer's instructions. XIAP (Human) Cell-Based ELISA Kit (Abnova, Taiwan), Alpha Ketoglutarate (alpha KG) Assay Kit (ab83431) (Abcam, France) Human FTO ELISA Kit (68ELH-FTO) (Tebu-Bio, France) Methyltransferase like 3 (METTL3), ELISA Kit (MBS9326769) (My BioSource, USA), CST-PathScan® Total Ezh2 Sandwich ELISA Kit (Ozyme, France), EpiQuik Dnmt1 Assay Kit (EpiQuik Dnmt1 Assay Kit, Euromedex/EpiGentek, France), Human Bcl-2 ELISA Kit (Abcam, France), Caspase-2 ELISA Kit (Tebu-Bio, France) and PathScan® Total PD-L1 Sandwich ELISA Kit (Ozyme, France) were performed according to the manufacturer's instructions.

Tumor Xenografts in Nude Mice

Cells were harvested by trypsinization, washed and resuspended in saline buffer. Cell suspensions were injected s.c. into the flank of 7-8-week-old mice (Janvier, France) in 100 μl of sterile PBS. Tumor volume based on caliper measurements was calculated using the modified ellipsoidal formula (Tumor volume=½(length×width2)).

The experimental procedures with animals were in accordance with the guidelines of Institutional Animal Care and the French National Committee of Ethics. In addition, all experiments were conducted according to the Regulations for Animal Experimentation at the “Plateforme Animalerie” in the “Institut de Recherche en Sante de l'Université de Nantes (IRS-UN)” and approved by the French National Committee of Ethics.

Cell Lines

U87, U87IDH1mut, RN33b and A549 cells were obtained from the American Type Culture Collection (ATCC, Molsheim, France). HASTR040/astrocytes were obtained from Clonexpress (Gaithersburg, USA). OE21 cells were obtained from Sigma (France). HEP10 cells were obtained from ThermoFisher (France). MCF7 and T47D cells were provided by the Dr P. Juin's lab. SKOV3 cells were provided by the Dr E. Scottet's lab. OV90 cells were provided by the Dr R. Spisek's lab.

Results

The m6A Methyltransferase METTL3, the m6A Demethylase FTO and Alpha-Ketoglutarate Regulate the N6-Adenosine Methylation of miR-200b-3p

Literature reports that miR-200 and particularly miR-200b-3p play a role in GBM [17][18][19][20]. Berulava et al. (2015) have identified the presence of m6A in certain miRNAs such as miR-200b-3p [6]. In agreement with these findings, we have investigated the miR-200b-3p level expression (miR-200b-3pexp) and the percentage of miRNA-200b-3p containing m6A (miR-200b-3p % m6A) in a collection of 32 GBM samples. RT-qPCR experiments indicated a high level of heterogeneity in miR-200b-3pexp with a max/min ratio equal to 37.6 (data not shown). RNA immunoprecipitation performed with an anti-m6A antibody followed by qPCR analysis (miRIPm6A-qPCR) indicated that 10/32 tumors contained a miR-200b-3p % m6A>10% (data not shown). In addition, we observed a correlation between miR-200b-3p % m6A and miR-200b-3pexp (p=0.0022) (data not shown).

In order to identify the molecular mechanisms governing the N6-adenosine methylation of miR-200b-3p in GBM patients, we first focused our analyses on FTO and αKG, since FTO is an adenosine demethylase that requires alpha-ketoglutarate (αKG) to catalyze the adenosine demethylation [11]. In our collection of 32 GBMs, Pearson's correlation tests show an absence of significant correlation FTO expression level with miR-200b-3p % m6A (p=0.0824) (data not shown) and between αKG and miR-200b-3p % m6A (p=0.0668) (data not shown). To consider these two parameters, we isolated GBM samples harboring a low FTO expression level (lower than median) and a low αKG level (lower than median) (FTOLow/αKGLow) from the other GBM samples (data not shown). Based on this subdivision, we noted that GBM samples harboring FTOLow/αKGLow were more m6A-methylated than other GBM samples (p=0.0042) (data not shown). Thus, we conclude that both FTO and αKG affect the m6A-methylation level of miR-200b-3p: the N6-adenosine methylation level of miR-200b-3p is elevated when FTO and αKG levels are lower. The involvement of FTO and αKG in the N6-adenosine methylation of miR was also supported by the fact that siRNA directed against FTO increased miR-200b-3p % m6A (data not shown), αKG treatment decreased miR-200b-3p % m6A (data not shown), Meclofenamic Acid (MA, a selective FTO inhibitor [21]) increased the miR-200b-3p % m6A (data not shown). In addition, we noted that the knock-down of ALKBH5 (a RNA adenosine demethylase [10]) did not changed the miR-200b-3p % m6A (data not shown). Thus, all these results support the idea that FTO and αKG act in concert to decrease the adenosine methylation of miR-200b-3p.

Alarcón et al. (2015) having identified that methyltransferase-like 3 (METTL3) methylates pri-miRNA in mammalian cells [5], we hypothesized that METTL3 could be implicated in the adenosine methylation of miR-200b-3p. To support this hypothesis, we first observed a significant correlation between miR-200b-3p % m6A and the METTL3 expression level (p=0.0010) (data not shown). Secondly, acellular experiments indicated that the immunoprecipitate of METTL3 (i.e. METTL3-including complexes) methylates miRNA-200b-3p in vitro (data not shown). Thirdly, METTL3 knock-down (siRNA method) decreased the level of m6A in miR-200b-3p (data not shown). To conclude, these three distinct experiments implicate METTL3 as a writer of N6-adenosine methylation of miR-200b-3p.

All the above results suggest that αKG, FTO and METTL3 collectively influence the presence of m6A in miR-200b-3p. In order to take into consideration the influence of these three parameters on the level of adenosine methylation of miR-200c-3p, we have calculated what we called the αFMscore. For each GBM samples, +1 was affected when the expression of αKG, FTO and METTL3 is predicted to increase the N6-adenosine methylation i.e. when the αKG and FTO expressions are lower or equal to the median value of our cohort and when METTL3 expression is higher than the median value of our cohort. −1 was affected when the expression of αKG, FTO and METTL3 is predicted to decrease the N6-adenosine demethylation i.e. when the αKG and FTO expressions are higher than the median value of our cohort and when METTL3 expression is lower or equal to the median value of our cohort. For example, a GBM harboring a high level of αKG and FTO and a low level of METTL3 has a αFMscore equal to +1, while another GBM harboring a low level of αKG and FTO and a low level of METTL3 has a αFMscore equal to +3. Thus, we noted that the αFMscore and the percentage of presence of m6A in miR-200b-3p were significantly correlated in our collection of 32 GBM (p=0.0006) (data not shown).

Taken together, our data support the idea that METTL3, FTO and αKG are involved in the regulation of the N6-adenosine methylation of miR-200b-3p.

The N6-Adenosine Methylation of miR-200b-3p Limits its Translational Repressor Function Towards Anti-Apoptotic Players and Confers Poor Prognosis in GBM Patients

XIAPmRNA being identified as a target of miR-200b-3p (according to the miRTarBase website), we next investigated whether there is a link between miR-200b-3pexp, miR-200b-3p % m6A and the XIAP expression in our collection of 32 GBM samples.

Our study did not correlate miR-200b-3pexp and the XIAP expression when all GBM samples were considered (p=0.8803) (data not shown).

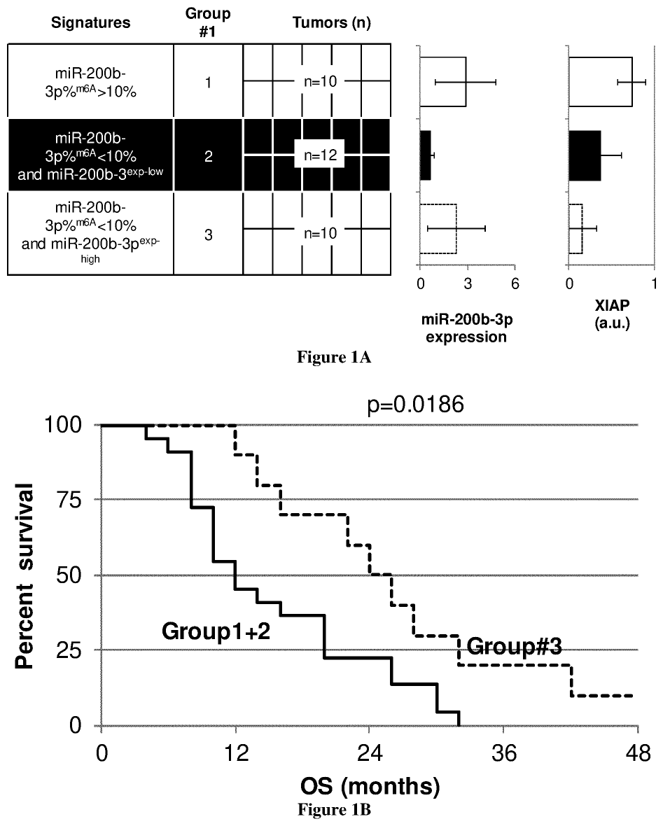

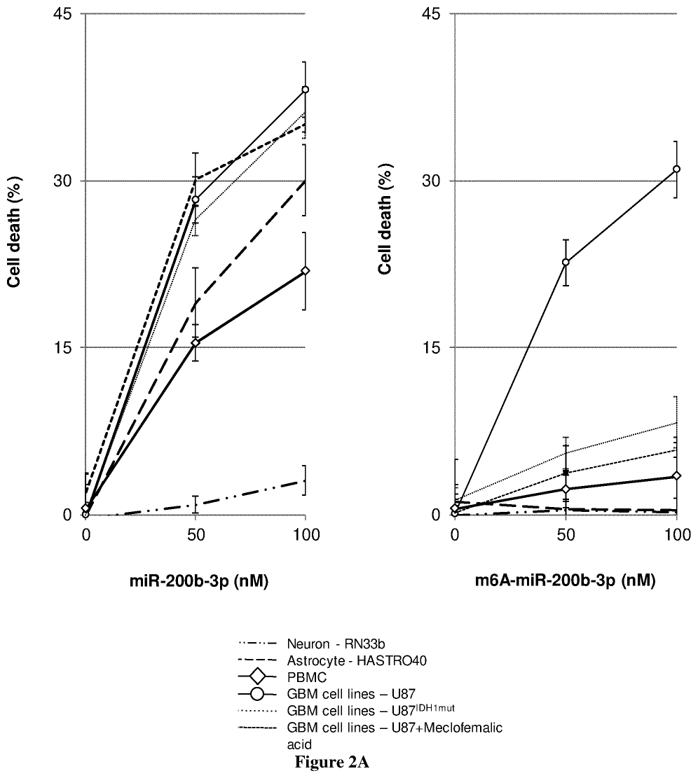

We then extended our study by dividing our samples in 3 groups by taking into consideration the adenosine methylation percentage of miR-200b-3p (FIG. 1A). Group #1 included samples with miR-200b-3p % m6A>10%. Group #2 included samples with a percentage miR-200b-3p % m6A<10% and miR-200b-3pexp inferior to the median (miR-200b-3pexp-low). Group #3 included samples with miR-200b-3p % m6A<10 and an expression level of miR-200b-3p superior to the median (miR-200b-3pexp-high).

For all samples having miR-200b-3p % m6A<10 (group #2 and #3), we noted that XIAP expression is inversely correlated with miR-200b-3pexp (FIG. 1A). This data is consistent with the dogma saying that miRNA is a post-transcriptional repressor.

Surprisingly, we noted that the average of XIAP expression of group #1's samples is higher than the ones of the two other groups (FIG. 1A). These results suggest that miR-200b-3p regulates XIAP expression when its sequence does not contain m6A (or a level inferior to 10%) and that the m6A presence in miR-200b-3p could abrogate the post-transcriptional repressor function of this miRNA.

To investigate this hypothesis, U251 cells were treated with an unspecific oligonucleotide (negative control), miR-200b-3pmimetic or m6A-modified miR-200b-3pmimetic. As expected, we did not observe any change in XIAP expression when cells were treated with unspecific oligonucleotide, while XIAP expression strongly decreased when cells were treated with miR-200b-3pmimetic (data not shown). Interestingly, we noted that this decrease is less efficient when cells were treated with the same quantity of m6A-modified miR-200b-3pmimetic (data not shown). Thus, it appears that the presence of m6A in miR-200b-3p abrogates the post-transcriptional repressor function of this miRNA toward XIAPmRNA.

We next performed Cross-Linking Immunoprecipitation and qPCR (CLIP-qPCR) analyses to determine whether the adenosine-methylation of miR-200b-3p influences the endogenous formation of 3′UTR-mRNA-XIAP/miR-200b-3p duplex. In our assays, immunoprecipitation is performed via an antibody directed against GW182 and TNRC6B (i.e. two proteins of the RISC complex having a central role in miRNA-mediated silencing), and qPCRs were performed to detect the enrichment/presence of miRNA and 3′UTRmRNA on the GW182- and TNRC6B-mediated co-immunoprecipitation products. CLIP-qPCRs were performed from samples with knock-down of METTL3 in order to estimate the impact of the loss of adenosine-methylation on the GW182- and TNRC6B-mediated co-immunoprecipitation of miRNAs and mRNAs. The miR-150-5p/3′UTR-mRNA-EP300 duplex was considered as a control. The choice of this control was dictated by the fact that miR-150-5p is not adenosine-methylated and the fact that miR-150-5p targets 3′UTR-mRNA-EP300.

We first noted that miR-150-5p and 3′UTR-mRNA-EP300 were present in GW182- and TNRC6B-mediated co-immunoprecipitation products, and this independently of the METTL3 knock-down (data not shown). Secondly, we noted that the METTL3 knock-down increased the presence of miR-200b-3p and 3′UTR-XIAP in the GW182- and TNRC6B-immunoprecipitates (data not shown). Thus, these last results indicate that the METTL3-mediated adenosine-methylation status of miR-200b-3p influences the endogenous formation of 3′UTR-mRNA-XIAP/miR-200b-3p duplex.

By affecting the expression of XIAP, an apoptotic player, our data suggest that the expression level and the N6-adenosine methylation level of miR-200b-3p could affect the intrinsic apoptosis level of tumors. To investigate this hypothesis, we analyzed the Caspase/DEVDase activity as a marker of the intrinsic apoptosis level of tumors. Our work indicates that tumors harboring the miRNA-200b-3pexp-low signature or the miR-200b-3p % m6A>10% signature have a lower intrinsic apoptosis level (data not shown).

Finally, we observed that patients whose tumors harbored the miRNA-200b-3pexp-low signature or the miR-200b-3p % m6A>10% signature have a lower survival outcome than the other GBM patients (FIG. 1B).

m6A-miR-200b-3p Appears as a Promising Tool in Anti-GBM Therapy

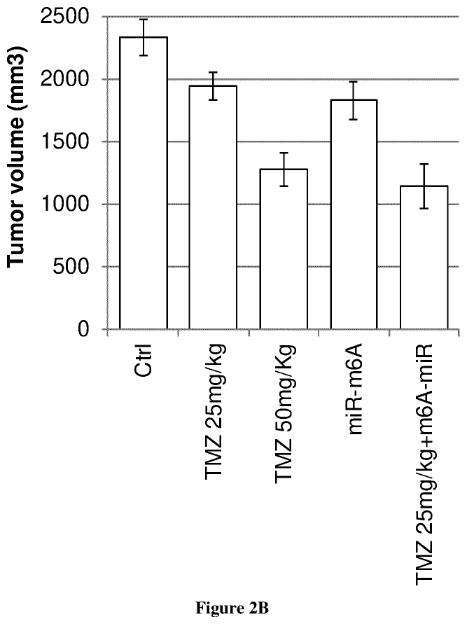

Based on the fact that the miR-200b-3p affects the intrinsic apoptosis level, we extended our study by investigating whether miR-200b-3p and m6A-miR-200b-3p could be used as a therapeutic tool. For this purpose, the miR-200b-3p- and m6A-miR-200b-3p-induced cell death was measured from a panel of cells representing human brain cells (astrocytes (HAST40), neurons (RN33b) and astrocytoma (U87). We included in this panel U87IDH1mut cells since IDH1 mutation is observed in GBM. Besides, we observed that the presence of IDH1 mutation decreased αKG and increased the adenosine methylation of miR-200b-3p in a context of the FTO and METTL3 expression level being unchanged (data not shown). Meclofemalic acid was also used as a FTO inhibitor [21]. Because peripheral blood is the place where exposure to chemicals occurs, PBMC (peripheral blood mononuclear cells) were also included in our study. Firstly, our data indicated that miRNA-200b-3p induced cell death in all cells with the exception of neuron (RN33b cell line) (FIG. 2A). Secondly, we observed that m6A-miR-200b-3p induced cell death in U87 cells, but not in U87IDH1mut, U87Meclofemalic, PBMC, neurons and astrocytes (FIG. 2A). In other terms, these data suggest that the ability of m6A-miR-200b-3p to induce cell death occurs in cancer cells and not in non-cancerous cells like PMBC, neurons and astrocytes. Based on our knowledge, the absence of massive m6A-miR-200b-3p-induced cell death in U87IDH1mut could be associated to the fact that these cells have a lower quantity of αKG, i.e. a lower quantity of the enzyme co-factor (FTO) catalyzing the adenosine demethylation of miR-200b-3p. Besides, the fact that the meclofemalic acid treatment abrogated the m6A-miR-200b-3p-induced cell death in U87 cells confirmed the involvement of FTO in this process (FIG. 2A).

We have then investigated the putative anti-GBM effect of m6A-miR-200b-3p in an in vivo model of GBM. For this purpose, U87-induced GBMs were generated by xenograft in mice. When the volume of the U87-induced GBMs was close to 100 mm3, three mice were randomly untreated, treated with temozolomide (TMZ) and/or with m6A-miR-200b-3p (data not shown). The option to use TMZ is due to the fact that this alkylating agent is the chemotherapeutic agent included in the current standard care protocol in GBM treatment [22].

By comparing the effect of the TMZ treatment with the effect of the m6A-miR-200b-3p treatment, we could clearly see that the m6A-miR-200b-3p treatment has similar efficiency than the TMZ-25 mg/kg treatment (FIG. 2B). We also noted that the m6A-miR-200b-3p+TMZ-25 mg/kg treatment has the same efficiency than the TMZ-50 mg/kg treatment (FIG. 2B).

miR-200b-3p could Also be Used as a Therapeutic Tool in Other Cancer Types

The above data are focused on the XIAP regulation by miR-200b-3p, but it is well known that one miRNA has multiple targets. Consequently, we next investigated whether the adenosine methylation of miR-200b-3p could abrogate its translational repressor function towards other putative protein targets than XIAP. Among the putative protein targets of miR-200b-3p (according to the miRTarBase website [23]), we focused our study on two other apoptotic players (Bcl-2 (B-cell lymphoma 2, Uniprot #P10415) and Caspase-2 (cysteine-dependent aspartate-directed proteases 2, Uniprot #P42575)), two epigenetic players (EZH2 (Enhancer of zeste homolog 2, Uniprot #Q15910) and DNMT1 (DNA (cytosine-5)-methyltransferase 1, Uniprot #P26358)) and a negative immune checkpoint PD-L1 (Programmed cell death 1 ligand 1, Uniprot #Q2NZQ7). Our data indicated that the presence of m6A in miRNA-200b-3p also abrogated the translational repressor function of miR-200b-3p toward Bcl-2 and PD-L1 (data not shown).

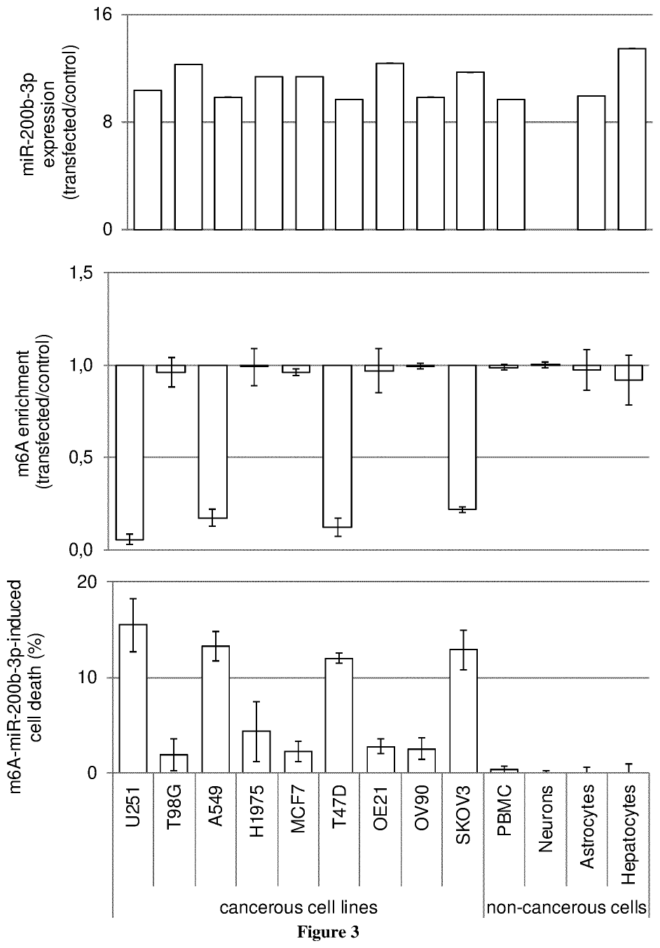

Finally, we investigated whether the ability of m6A-miR-200b-3p to induce cell death was specific of U87 cells. For this purpose, cancerous cell lines representative of several cancers were transfected with m6A-miR-200b-3p (U251 and T98G for glioblastoma, A549 and H1975 for lung, MCF7 and T47D for breast, OE21 for esophagus, OV90 and SKOV3 for ovaries). Four non-cancerous cell lines were also included in our study. Four hours after cells transfection, we noted that all cells were transfected with similar quantity of m6A-miR-200b-3p since the range of increase of miR-200b-3p expression was homogeneous (10-13 fold induction) (FIG. 3). Then, we noted that cell death occurred in cells having the ability to adenosine-demethylate miR-200b-3b i.e. in U251, A549, T47D and SKOV3 (FIG. 3). The absence of cell death in other cell lines and particularly in non-cancerous cell lines was explained by the inability of these cells to adenosine-methylate miR-200b-3b (m6A enrichment transfected/control being equal to 1) (FIG. 3).

Taken together, all these last results are consistent with the fact that m6A-miR-200b-3p appears as a promising tool in anti-GBM therapy.

Conclusion:

Recent investigations concerning the description of the molecular mechanisms of bases modification of miRNAs have provided meaningful progresses in the understanding of regulation of the miRNAs biogenesis and functionality. Thus, after the studies of Alarcón et al. (2015), Berulava et al. (2015) and Konno et al. (2019), our study reports the presence of m6A in miRNAs via the realization of RNA immunoprecipitation with an anti-m6A-antibody followed by RT-qPCR [5] [6] [7]. Despite these posterior studies, our investigation harbors several innovative points.

First, the work of the inventors indicates that the adenosine methylation of miR-200b-3p abrogates its translational repressor function towards its putative targets such as XIAP, Bcl-2 and PD-L1. The works published by Alarcón et al. (2015) and Berulava et al. (2015) report the existence of 2 different consensus sequences for the m6A methylation in pri-miRNAs (UGAC) and in mature miRNAs (ADRA) [5][6]. Interestingly, the inventors noted that the miRNA-200b-3p sequence contains a sequence matching one of the consensus. They also noted that the miR-200b-3p sequence contains a sequence matching the consensus sequence binding by METTL3/WTAP defined by Ping et al. (2014) [12]. From a certain perspective, this last point can also constitute an argument supporting the role of METTL3 in the adenosine methylation of miRNAs.

The work of Berulava et al. (2015) indicate that FTO plays a crucial role in the demethylation of miRNAs [6]. The data of the inventors complete this by indicating that the presence of αKG also acts as a non-negligible player in the demethylation of miRNAs.

In addition to these 2 initial reports, this study shows that the presence of m6A acts as an inhibitor of the post-transcriptional repressor function of miRNAs. Mechanistically, these data indicate that the presence of m6A limits the formation of miRNA/mRNA duplex. This study is also distinguished from the first two studies by its clinical translational study effort using a cohort of cancer patients. Indeed, this study is the first to mention that the level of N6-adenosine methylation of a miRNA (in association with the expression level of this miRNA) acts as a biomarker characterizing GBM patients with a poor survival. This study is also distinct to the one recently published by Konno et al. (2019) since Konno and colleagues considerate the adenosine methylation of miR as a tool to distinguish early pancreatic cancer patients from healthy controls with an extremely high sensitivity and specificity; while in our article the adenosine methylation of miR-200b-3p is associated with a prognosis value of response for GBM patients [7] and could have a therapeutic function.

The work of Berulava et al. (2015) and the one of Yuan et al. (2014) introduce a debate about the impact of the adenosine methylation of miRNA on their stability [6] [24]. These data focusing on miR-200b-3p seems to indicate that the adenosine methylation of this miRNA does not impact on its expression. Indeed, the modulation of its adenosine methylation level via siRNA directed against FTO and METTL3 or via chemical components does not affect its expression. However, this finding being obtained on one miRNA, it is not possible to generalize a rule about the impact of the adenosine methylation on the miRNA stability.

By observing that the adenosine methylated miR-200b-3p was not recruited to the RISC complex, these data reinforces the idea that the adenosine methylation of miRNA appears as molecular mechanism governing the miRNA functionality via the regulation of the duplex formation between miRNA and mRNA. More generally, these data support the idea that nucleotide modification occurring in miRNA or in 3′UTR-mRNA alters the formation of miR/3′UTR-mRNA duplex, such as reported by Lockhart et al. (2019) [25].

By reporting that m6A methylation of miRNAs could act as a biomarker characterizing GBM patients with a poor survival, our data open the idea that the molecular actor writing this epitranscriptomic signature (METTL3 according to our data) could be used as a target for the development of epidrugs. Indeed, this point of view is already discussed since METTL3 promotes oncogene translation [26].

During the last decade, miRNA mimics and molecules targeting miRNAs (anti-miRs) have shown promising results in preclinical development [27][28]. Four arguments strongly support the idea that the adenosine-methylated form of miR-200b-3p could be used as a promising therapeutic tool. First, m6A-miR-200b-3p is apoptogenic by itself via the repression of XIAP, an anti-apoptotic protein. Secondly, these data indicate that m6A-miR-200b-3p promotes cell death in cancerous cells such as U87 (but also in other cancer cell lines) and not in non-cancerous cells such as neurons, PBMC, astrocytes and hepatocytes. Thirdly, the in vivo data of the inventors indicate that m6A-miR-200b-3p has an anti-tumor growth effect in an in vivo model of GBM. Fourthly, these in vivo data also indicate that the m6A-miR-200b-3p/TMZ combination permits to limit the dose of TMZ since the m6A-miR-200b-3p/TMZ-25 mg/kg combination has the same anti-tumor growth effect than the use of the TMZ-50 mg/kg treatment. Thus, all these arguments define the adenosine-methylated form of miR-200b-3p as the prodrug form of this miRNA. More interestingly, these data indicate that its conversion under an active form occurs in cancer cells but not in non-cancerous cells. This observation is highly promising since it can be translated such as the fact that only cancerous cells have the “tools” (FTO and αKG) to activate the prodrug form of miR-200b-3p. Thus, the adenosine-methylated form of miRNAs could be considered such as a manner to limit the off-targets effect of miRNA therapy associated with the relative lack of addressing of miRNA-based therapy against the cancer cells [29]. These data also introduce the idea that the presence of IDH1 mutations could be considered such as a biomarker excluding the use of adenosine-methylated form of miRNAs since cells presenting IDH1 mutations have a low level of αKG. Concretely, the first reading of this idea might exclude the use of m6A-miR-200b-3p treatment in less than 10% of primary GBM and in 6-10% of de novo AML, as example [30] [31]. However, this point is available when the m6A-miR-200b-3p treatment is envisioned as single treatment since its combination with BAY1436032 (a pan-mutant IDH1 inhibitor [32]) restored its ability to promote cell death (data not shown).

In conclusion, the results of the inventors opens a new area in the understanding of epigenetic modifications concerning miRNA and in the development of innovative epidrugs. Indeed, since several years chemical modifications of RNAs (i.e. epitranscriptomic) are defined such as central players in the control of messenger and ncRNA activity [33]. Our data reinforce this idea by showing that the adenosine methylation of miRNAs abrogates their post-transcriptional repressive function. By initiating the idea that adenosine-methylated miRNA could be used as a prodrug, our work provides the base for the development of a new pathway of anti-cancer therapeutic strategies targeting miRNA. Thus, in the future years, the understanding of the mechanisms involved in the epigenetic regulation of miRNA could improve patient stratification and the development of successful miRNA-based therapeutic strategies.

### REFERENCES

Throughout this application, various references describe the state of the art to which this invention pertains. The disclosures of these references are hereby incorporated by reference into the present disclosure.
[1] Wang Z, Yao H, Lin S, Zhu X, Shen Z, Lu G, et al. Transcriptional and epigenetic regulation of human microRNAs. Cancer Lett 2013; 331:1-10. https://doi.org/10.1016/j.canlet.2012.12.006.
[2] Van den Hove D L, Kompotis K, Lardenoije R, Kenis G, Mill J, Steinbusch H W, et al. Epigenetically regulated microRNAs in Alzheimer's disease. Neurobiol Aging 2014; 35:731-45. https://doi.org/10.1016/j.neurobiolaging.2013.10.082.
[3] Malumbres M. miRNAs and cancer: an epigenetics view. Mol Aspects Med 2013; 34:863-74. https://doi.org/10.1016/j.mam.2012.06.005.
[4] Xhemalce B, Robson S C, Kouzarides T. Human RNA methyltransferase BCDIN3D regulates microRNA processing. Cell 2012; 151:278-88. https://doi.org/10.1016/j.cell.2012.08.041.
[5] Alarcón C R, Lee H, Goodarzi H, Halberg N, Tavazoie S F. N6-methyladenosine marks primary microRNAs for processing. Nature 2015; 519:482-5. https://doi.org/10.1038/nature14281.
[6] Berulava T, Rahmann S, Rademacher K, Klein-Hitpass L, Horsthemke B. N6-adenosine methylation in MiRNAs. PLoS ONE 2015; 10:e0118438. https://doi.org/10.1371/journal.pone.01 18438.
[7] Konno M, Koseki J, Asai A, Yamagata A, Shimamura T, Motooka D, et al. Distinct methylation levels of mature microRNAs in gastrointestinal cancers. Nat Commun 2019; 10:3888. https://doi.org/10.1038/s41467-019-11826-1.
[8] Pandolfini L, Barbieri I, Bannister A J, Hendrick A, Andrews B, Webster N, et al. METTL1 Promotes let-7 MicroRNA Processing via m7G Methylation. Mol Cell 2019; 74:1278-1290.e9. https://doi.org/10.1016/j.molcel.2019.03.040.
[9] Cheray M, Etcheverry A, Jacques C, Pacaud R, Bougras-Cartron G, Aubry M, et al. Cytosine methylation of mature microRNAs inhibits their functions and is associated with poor prognosis in glioblastoma multiforme. Mol Cancer 2020; 19:36. https://doi.org/10.1186/s12943-020-01155-z.
[10] Xu C, Liu K, Tempel W, Demetriades M, Aik W, Schofield C J, et al. Structures of human ALKBH5 demethylase reveal a unique binding mode for specific single-stranded N6-methyladenosine RNA demethylation. J Biol Chem 2014; 289:17299-311. https://doi.org/10.1074/jbc.M114.550350.
[11] Gerken T, Girard C A, Tung Y-C L, Webby C J, Saudek V, Hewitson K S, et al. The obesity-associated FTO gene encodes a 2-oxoglutarate-dependent nucleic acid demethylase. Science 2007; 318:1469-72. https://doi.org/10.1126/science.1151710.
[12] Ping X-L, Sun B-F, Wang L, Xiao W, Yang X, Wang W-J, et al. Mammalian WTAP is a regulatory subunit of the RNA N6-methyladenosine methyltransferase. Cell Res 2014; 24:177-89. https://doi.org/10.1038/cr.2014.3.
[13] Balacco D L, Soller M. The m6A Writer: Rise of a Machine for Growing Tasks. Biochemistry 2019; 58:363-78. https://doi.org/10.1021/acs.biochem.8b01166.
[14] Zdzisinska B, Zurek A, Kandefer-Szerszen M. Alpha-Ketoglutarate as a Molecule with Pleiotropic Activity: Well-Known and Novel Possibilities of Therapeutic Use. Arch Immunol Ther Exp (Warsz) 2017; 65:21-36. https://doi.org/10.1007/s00005-016-0406-x.
[15] Scott F L, Denault J-B, Riedl S J, Shin H, Renatus M, Salvesen G S. XIAP inhibits caspase-3 and -7 using two binding sites: evolutionarily conserved mechanism of IAPs. EMBO J 2005; 24:645-55. https://doi.org/10.1038/sj.emboj.7600544.
[16] Li S, Pan B, Li L, Shi B, Xie F, He C. Prognostic significance of X-linked inhibitor of apoptosis protein in solid tumors: A systematic review and meta-analysis. J Cell Physiol 2019; 234:18111-22. https://doi.org/10.1002/jcp.28443.
[17] Liu J, Wang L, Li X. HMGB3 promotes the proliferation and metastasis of glioblastoma and is negatively regulated by miR-200b-3p and miR-200c-3p. Cell Biochem Funct 2018; 36:357-65. https://doi.org/10.1002/cbf.3355.
[18] Peng L, Fu J, Ming Y. The miR-200 family: multiple effects on gliomas. Cancer Manag Res 2018; 10:1987-92. https://doi.org/10.2147/CMAR.S160945.
[19] Men D, Liang Y, Chen L. Decreased expression of microRNA-200b is an independent unfavorable prognostic factor for glioma patients. Cancer Epidemiol 2014; 38:152-6. https://doi.org/10.1016/j.canep.2014.01.003.
[20] Peng B, Hu S, Jun Q, Luo D, Zhang X, Zhao H, et al. MicroRNA-200b targets CREB1 and suppresses cell growth in human malignant glioma. Mol Cell Biochem 2013; 379:51-8. https://doi.org/10.1007/s11010-013-1626-6.
[21] Huang Y, Yan J, Li Q, Li J, Gong S, Zhou H, et al. Meclofenamic acid selectively inhibits FTO demethylation of m6A over ALKBH5. Nucleic Acids Res 2015; 43:373-84. https://doi.org/10.1093/nar/gkul276.
[22] Fernandes C, Costa A, Os6rio L, Lago R C, Linhares P, Carvalho B, et al. Current Standards of Care in Glioblastoma Therapy. In: De Vleeschouwer S, editor. Glioblastoma, Brisbane (AU): Codon Publications; 2017.
[23] Chou C-H, Shrestha S, Yang C-D, Chang N-W, Lin Y-L, Liao K-W, et al. miRTarBase update 2018: a resource for experimentally validated microRNA-target interactions. Nucleic Acids Res 2018; 46:D296-302. https://doi.org/10.1093/nar/gkx1067.
[24] Yuan S, Tang H, Xing J, Fan X, Cai X, Li Q, et al. Methylation by NSun2 represses the levels and function of microRNA 125b. Mol Cell Biol 2014; 34:3630-41. https://doi.org/10.1128/MCB.00243-14.
[25] Lockhart J, Canfield J, Mong E F, VanWye J, Totary-Jain H. Nucleotide Modification Alters MicroRNA-Dependent Silencing of MicroRNA Switches. Mol Ther Nucleic Acids 2019; 14:339-50. https://doi.org/10.1016/j.omtn.2018.12.007.
[26] Lin S, Choe J, Du P, Triboulet R, Gregory R I. The m(6)A Methyltransferase METTL3 Promotes Translation in Human Cancer Cells. Mol Cell 2016; 62:335-45. https://doi.org/10.1016/j.molcel.2016.03.021.
[27] Rupaimoole R, Slack F J. MicroRNA therapeutics: towards a new era for the management of cancer and other diseases. Nat Rev Drug Discov 2017; 16:203-22. https://doi.org/10.1038/nrd.2016.246.
[28] Tutar L, Tutar E, Ozg0r A, Tutar Y. Therapeutic Targeting of microRNAs in Cancer: Future Perspectives. Drug Dev Res 2015; 76:382-8. https://doi.org/10.1002/ddr.21273.
[29] Chen Y, Zhao H, Tan Z, Zhang C, Fu X. Bottleneck limitations for microRNA-based therapeutics from bench to the bedside. Pharmazie 2015; 70:147-54.
[30] Dang L, Yen K, Attar E C. IDH mutations in cancer and progress toward development of targeted therapeutics. Ann Oncol 2016; 27:599-608. https://doi.org/10.1093/annonc/mdw013.
[31] Mondesir J, Willekens C, Touat M, de Botton S. IDH1 and IDH2 mutations as novel therapeutic targets: current perspectives. J Blood Med 2016; 7:171-80. https://doi.org/10.2147/JBM.S70716.
[32] Pusch S, Krausert S, Fischer V, Balss J, Ott M, Schrimpf D, et al. Pan-mutant IDH1 inhibitor BAY 1436032 for effective treatment of IDH1 mutant astrocytoma in vivo. Acta Neuropathol 2017; 133:629-44. https://doi.org/10.1007/s00401-017-1677-y.
[33] Esteller M, Pandolfi P P. The Epitranscriptome of Noncoding RNAs in Cancer. Cancer Discov 2017; 7:359-68. https://doi.org/10.1158/2159-8290.CD-16-1292.
[34] Cartron P-F, Oliver L, Martin S, Moreau C, LeCabellec M-T, Jezequel P, et al. The expression of a new variant of the pro-apoptotic molecule Bax, Baxpsi, is correlated with an increased survival of glioblastoma multiforme patients. Hum Mol Genet 2002; 11:675-87.

1. An in vitro method for determining the prognosis of the survival time of a patient suffering from a cancer and then treating the patient comprising i) determining the expression level of miR-200b-3p and/or N6-adenosine methylated miRNA-200b-3p (miR-200b-3p m6A) in a sample from said patient, ii) determining that said expression level of miR-200b-3p m6A is inferior to 10% of a miR-200b-3p m6A predetermined reference value and/or that the expression level of the miR-200b-3 is higher than an miR-200b-3 predetermined reference value and iii) treating the patient determined to have an expression level of miR-200b-3p m6A that is inferior to 10% of the miR-200b-3p m6A predetermined reference value and/or an expression level of miR-200b-3 that is higher than the miR-200b-3 predetermined reference value with a therapeutically effective amount of N6-adenosine methylated miRNA-200b-3p (miR-200b-3p m6A).

2. The in vitro method according to the claim 1 wherein the expression level of the miR-200b-3p and the expression level of N6-adenosine methylated miRNA-200b-3p (miR-200b-3p m6A) are both determined.

3. The in vitro method according to claim 1 wherein the cancer is a glioblastoma multiforme (GBM).

4. The in vitro method according to claim 1, wherein the sample according to the invention is blood, plasma, serum sample or a cancer biopsy.

5. (canceled)

6. (canceled)

7. (canceled)

8. (canceled)

9. A method for treating cancer comprising administering to a subject in need thereof a therapeutically effective amount of N6-adenosine methylated miRNA-200b-3p (miR-200b-3p m6A).

10. (canceled)

11. The method of claim 9, wherein the N6-adenosine methylated miRNA-200b-3p (miR-200b-3p m6A) is a prodrug.

12. The method of claim 9, wherein the cancer is a glioblastoma multiforme (GBM).

13. The method of claim 9, wherein the miR-200b-3p m6A has the nucleic acid sequence SEQ ID NO:1 with a methylation on the second to last nucleic acid of the 3′ end.


SEQUENCE LISTING (1 sequence)

SEQ ID NO: 1 - RNA, 22 nt, Homo sapiens
uaauacugccugguaaugauga